(19) United States
(12) Patent Application Publication
(10) Pub. No.: US 2023/0000956 A1
(43) Pub. Date: Jan. 5, 2023

(54) COMBINATION THERAPY WITH GLUCARPIDASE WITH METHOTREXATE/RITUXIMAB TO TREAT CNS LYMPHOMA

(71) Applicants: MEMORIAL SLOAN KETTERING CANCER CENTER, New York, NY (US); UAB RESEARCH FOUNDATION, Birmingham, AL (US)

(72) Inventors: Lauren SCHAFF, New York, NY (US); Christian GROMMES, New York, NY (US); Lisa DEANGELIS, New York, NY (US); Burt NABORS, Birmingham, AL (US)

(21) Appl. No.: 17/767,371

(22) PCT Filed: Oct. 7, 2020
(86) PCT No.: PCT/US2020/054569

§ 371 Date: Apr. 7, 2022

Related U.S. Application Data
(60) Provisional application No. 62/912,424, filed on Oct. 8, 2019.

(51) Int. Cl. A61K 38/48; A61K 31/519; A61K 39/395; A61K 45/06; A61P 35/00

(52) U.S. Cl. CPC A61K 38/4813; A61K 31/519; A61K 39/3955; A61K 45/06; A61P 35/00

(57) ABSTRACT

The present technology relates to methods comprising the administration of methotrexate and glucarpidase to treat central nervous system lymphoma in a subject in need thereof. Kits for use in practicing the methods are also provided.

### CROSS-REFERENCE TO RELATED APPLICATIONS

This application is a U.S. National Stage Application under 35 U.S.C. § 371 of International Patent Application No. PCT/US2020/054569, filed on Oct. 7, 2020, which claims the benefit of and priority to U.S. Provisional Patent Application No. 62/912,424, filed Oct. 8, 2019, the entire contents of which are incorporated herein by reference.

### SEQUENCE LISTING

The instant application contains a Sequence Listing which has been submitted electronically in ASCII format and is hereby incorporated by reference in its entirety. Said ASCII copy, created on Dec. 1, 2020, is named 115872-0786_SL.txt and is 3,879 bytes in size.

### TECHNICAL FIELD

The present technology relates to methods comprising the administration of methotrexate and glucarpidase to treat central nervous system lymphoma in a subject in need thereof. Kits for use in practicing the methods are also provided.

### BACKGROUND

The following description of the background of the present technology is provided simply as an aid in understanding the present technology and is not admitted to describe or constitute prior art to the present technology.

Central nervous system lymphoma (CNSL) is an aggressive malignancy that results in significant patient morbidity and mortality. Primary CNSL (PCNSL), an aggressive subtype of diffuse large B-cell lymphoma (DLBCL), is typically responsive to high dose methotrexate (HD-MTX), with overall response rates (ORR) of up to 74% in the upfront setting (Batchelor, T, et al., J Clin Oncol, 21(6): p. 1044-9 (2003)). Still, prognosis is poor with 20-30% cured. Ferreri, A. J., Blood 118(3): p. 510-22 (2011); Gavrilovic, I. T., et al., J Clin Oncol 24(28): p. 4570-4 (2006)). Patients with disease of the bone marrow, testicles, paranasal sinuses, bone, retroperitoneal lymph nodes and epidural space as well as those with extranodal disease are at increased risk of development of secondary CNSL (SCNSL). SCNSL carries a poor prognosis with a median survival of 1-5 months. See Grier, J. and T. Batchelor, Curr Oncol Rep, 7(1): p. 55-60 (2005).

High-dose methotrexate (MTX)-based regimens are the standard treatment for CNSL. The optimal dosing of MTX for use in CNSL has not been well defined. MTX is predominantly cleared via renal excretion (70-90%) with contribution from the hepatic system via conversion of MTX to 7-hydroxymethotrexate. Between 2-12% of adults can sustain acute kidney injury as a result of HD-MTX. See Widemann, B. C., et al., J Clin Oncol, 28(25): p. 3979-86 (2010). Acute kidney injury can in return result in delayed MTX clearance and toxic levels which can lead to systemic effects such as hepatic injury, pneumonitis, and bone marrow suppression. While these toxicities are uncommon (<10%), the low incidence is due in part to MTX dose reduction (required in 14-44% of patients) or premature cessation of therapy (5-8% of patients) in the setting of MTX-related acute kidney injury. See Jahnke, K. et al., Ann Oncol 16(3): p. 445-9 (2005).

Thus, there is an urgent need for methods that reduce the nephrotoxic risks associated with high-dose methotrexate therapy (HD-MTX), particularly in view of the ongoing COVID-19 pandemic, which has highlighted healthcare as a limited resource, with elective inpatient admissions particularly problematic.

### SUMMARY

In one aspect, the present disclosure provides a method for treating central nervous system lymphoma in a subject in need thereof comprising (a) administering to the subject an effective amount of methotrexate; and (b) administering to the subject an amount of glucarpidase that is sufficient to reduce the level of methotrexate in the subject's serum by >90%, wherein the glucarpidase is administered 10 to 48 hours following administration of methotrexate, and wherein steps (a)-(b) are performed for 1, 2, 3, 4, 5, 6, 7, 8 or more cycles. In another aspect, the present disclosure provides a method for reducing the toxicity associated with high dose methotrexate therapy in a subject in need thereof comprising (a) administering to the subject an effective amount of methotrexate; and (b) administering to the subject an amount of glucarpidase that is sufficient to reduce the level of methotrexate in the subject's serum by >90%, wherein the glucarpidase is administered 10 to 48 hours following administration of methotrexate, wherein steps (a)-(b) are performed for 1, 2, 3, 4, 5, 6, 7, 8 or more cycles, and wherein the subject suffers from or is diagnosed with central nervous system lymphoma. The central nervous system lymphoma may be primary CNSL (PCNSL) or secondary CNSL (SCNSL). In a further embodiment, the PCNSL has developed in one or more tissues selected from the group consisting of brain parenchyma, spinal cord, meninges, cerebrospinal fluid, and eyes.

Additionally or alternatively, in some embodiments of the methods disclosed herein, the effective amount of methotrexate is about 3 to 10 g/m². In certain embodiments, the effective amount of methotrexate is about 3 g/m², about 3.5 g/m², 4 g/m², about 4.5 g/m², 5 g/m², about 5.5 g/m², 6 g/m², about 6.5 g/m², 7 g/m², about 7.5 g/m², 8 g/m², about 8.5 g/m², about 9 g/m², about 9.5 g/m², or about 10 g/m².

In any and all embodiments of the methods disclosed herein, the subject is suffering from or is diagnosed with diffuse large B-cell lymphoma (DLBCL). In certain embodiments, the subject exhibits metastases in one or more tissue sites selected from the group consisting of bone marrow, testicles, paranasal sinuses, bone, retroperitoneal lymph nodes and epidural space. Additionally or alternatively, in some embodiments, the subject is suffering from CNSL relapse.

In any of the preceding embodiments of the methods disclosed herein, glucarpidase and methotrexate are administered sequentially, or separately. In certain embodiments of the methods disclosed herein, methotrexate is administered subcutaneously, intravenously, intraperitoneally, intra-articularly, intra-synovially, intrasternally, intrathecally, orally, topically, transmucosally, iontophoretically, or via intracranial injection. Additionally or alternatively, in some embodiments, glucarpidase is administered subcutaneously, intravenously, intraperitoneally, intra-articularly, intra-synovially, intrasternally, intrathecally, orally, topically, transmucosally, iontophoretically, or via intracranial injection. In some embodiments, the subject is human.

Additionally or alternatively, in some embodiments, steps (a)-(b) are performed for up to 10 cycles. In other embodiments, steps (a)-(b) are performed for 3-8 cycles. In any and all embodiments of the methods disclosed herein, the amount of glucarpidase in at least one cycle is about 1800 to 2200 units. Additionally or alternatively, in some embodiments, the amount of glucarpidase in at least one cycle is about 800 to 1200 units. In certain embodiments, the amount of glucarpidase in at least one cycle is about 800 units, about 850 units, about 900 units, about 950 units, about 1000 units, about 1050 units, about 1100 units, about 1150 units, about 1200 units, about 1250 units, about 1300 units, about 1350 units, about 1400 units, about 1450 units, about 1500 units, about 1550 units, about 1600 units, about 1650 units, about 1700 units, about 1750 units, about 1800 units, about 1850 units, about 1900 units, about 1950 units, about 2000 units, about 2050 units, about 2100 units, about 2150 units, or about 2200 units. In any of the preceding embodiments of the methods disclosed herein, the amount of glucarpidase in the first 2 to 4 cycles is about 1800 to 2200 units and the amount of glucarpidase after the first 2 to 4 cycles is about 800 to 1200 units. In certain embodiments, the amount of glucarpidase in the first 2 to 4 cycles is about 2000 units and the amount of glucarpidase after the first 2 to 4 cycles is about 1000 units.

Additionally or alternatively, in some embodiments, the methods of the present technology further comprise administering to the subject an effective amount of an additional therapeutic agent that targets CNSL. The additional therapeutic agent that targets CNSL may be an anti-CD20 antibody, an anti-CD19 antibody, a steroid (e.g., glucocorticoids), a chemotherapeutic agent, and any combination thereof. Examples of anti-CD20 antibodies include, but are not limited to rituximab (e.g., MabThera®, Rixathon® and Truxima®), ocrelizumab, obinutuzumab, veltuzumab, ofatumumab, ibritumomab tiuxetan, ¹³¹I tositumomab, AME-133v, PRO131921, TRU-015, and GA101. Examples of anti-CD19 antibodies include, but are not limited to Blinatumomab, GBR 401, Coltuximabravtansine, MOR208, MEDI-551, Denintuzumabmafodotin, Taplitumomabpaptox, XmAb 5871, MDX-1342, AFM11, and SAR3419 (huB4-DM4). Additionally or alternatively, in some embodiments, the at least one additional therapeutic agent is a chemotherapeutic agent selected from the group consisting of cyclophosphamide, carmustine, etoposide, bisulfan, vincristine, procarbazine, temozolomide, cytarabine, and thiotepa.

In any of the preceding embodiments of the methods disclosed herein, the anti-CD20 antibody is administered in one or more cycles with methotrexate and glucarpidase. In certain embodiments, each cycle occurs over (i) a three day period and (ii) comprises: administering to the subject about 300-600 mg/m² of anti-CD20 antibody on day 1, administering to the subject about 3-10 mg/m² methotrexate on day 2, and administering to the subject about 800-2200 units of glucarpidase on day three. In certain embodiments, the amount of anti-CD20 antibody administered to the subject on day 1 is about 300 mg/m², about 350 mg/m², about 400 mg/m², about 450 mg/m², about 500 mg/m², about 550 mg/m², or about 600 mg/m².

In any and all embodiments of the methods disclosed herein, the subject exhibits a delay in metastatic onset and/or tumor growth after administration of MTX and glucarpidase compared to that observed in an untreated control subject diagnosed with CNSL.

The present disclosure also provides kits comprising MTX, glucarpidase, and instructions for treating CNSL in accordance with any and all embodiments of the methods disclosed herein.

### BRIEF DESCRIPTION OF THE DRAWINGS

FIG. 1 shows a schematic of the therapeutic regimen administered to subjects in two distinct Cohorts (at 3 mg/m² and 6 mg/m² of methotrexate (MTX)).

FIG. 2 shows the treatment regimen for each of the 8 enrolled patients. The clinical responses of patients 1-4 are indicated.

FIG. 3 shows serum MTX concentrations following HD-MTX 3, or 6 g/m² before and after administration of glucarpidase.

FIG. 4 shows serum and CSF MTX concentrations following HD-MTX 3, 6, or 8 g/m² before and after administration of glucarpidase.

FIG. 5 shows the radiographic response with HD-MTX in combination with glucarpidase.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition (John Wiley and Sons, Inc., NY, 1999)).

The present disclosure provides a method for treating CNSL patients with repeated high doses of methotrexate in combination with repeated reduced-doses of glucarpidase, and more particularly in conjunction with rituximab. The methods of the present technology reduce methotrexate (MTX) serum concentrations to levels that are considered non-problematic for systemic toxicities whilst maintaining MTX cerebrospinal fluid concentrations at therapeutic levels. Other studies have raised concerns regarding the emergence of glucarpidase neutralizing antibodies and their potential effects on glucarpidase activity in patients receiving the MTX/glucarpidase therapeutic regimen. See Adamson et al., J. Clinical Oncology 10(8): 1359-1364 (1992). However, the present study shows that the emergence of glucarpidase antibodies in a subset of treated patients receiving repeated doses of glucarpidase did not appear to negatively impact the efficacy of the MTX/glucarpidase therapeutic regimen with respect to ameliorating CNS lymphoma. Indeed, the methods disclosed herein are capable of providing complete or near complete patient responses over a treatment regimen involving multiple MTX-rituximab administrations followed by glucarpidase within 12-48 hours.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including orally, intranasally, parenterally (intravenously, intramuscularly, intraperitoneally, or subcutaneously), intratumorally, or topically. Administration includes self-administration and the administration by another.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease or condition, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, “KPS” or “Karnofsky Performance Status” refers to a standard way of measuring the ability of cancer patients to perform ordinary daily tasks. See Karnofsky D, Burchenal J, The clinical evaluation of chemotherapeutic agents in cancer. In: MacLeod C, ed. EVALUATION OF CHEMOTHERAPEUTIC AGENTS. New York, N.Y.: Columbia University Press 191-205 (1949). The KPS scores range from 0 to 100. A higher score means the patient is better able to carry out daily activities. KPS may be used to determine a patient's prognosis, to measure changes in a patient's ability to function, or to decide if a patient could be included in a clinical trial.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20th edition, ed. A. Gennaro, 2000, Lippincott, Williams & Wilkins, Philadelphia, Pa.).

As used herein, “prevention,” “prevent,” or “preventing” of a disease or condition refers to one or more compounds that, in a statistical sample, reduces the occurrence of the disease or condition in the treated sample relative to an untreated control sample, or delays the onset of one or more symptoms of the disease or condition relative to the untreated control sample. As used herein, prevention includes preventing or delaying the initiation of symptoms of the disease or condition. As used herein, prevention also includes preventing a recurrence of one or more signs or symptoms of a disease or condition.

“RECIST” shall mean an acronym that stands for “Response Evaluation Criteria in Solid Tumors” and is a set of published rules that define when cancer patients improve (“respond”), stay the same (“stable”) or worsen (“progression”) during treatments. Response as defined by RECIST criteria have been published, for example, at Journal of the National Cancer Institute, Vol. 92, No. 3, Feb. 2, 2000 and RECIST criteria can include other similar published definitions and rule sets. One skilled in the art would understand definitions that go with RECIST criteria, as used herein, such as “Partial Response (PR),” “Complete Response (CR),” “Stable Disease (SD)” and “Progressive Disease (PD).”

The irRECIST overall tumor assessment is based on total measurable tumor burden (TMTB) of measured target and new lesions, non-target lesion assessment and new non-measurable lesions. At baseline, the sum of the longest diameters (SumD) of all target lesions (up to 2 lesions per organ, up to total 5 lesions) is measured. At each subsequent tumor assessment (TA), the SumD of the target lesions and of new, measurable lesions (up to 2 new lesions per organ, total 5 new lesions) are added together to provide the TMTB.

Overall Assessments by irRECIST

| | |
|---|---|
| Complete | Complete disappearance of all measurable |
| Response (irCR) | and non-measurable lesions. Lymph nodes must decrease to <10 mm |
| | in short axis. |
| Partial Response (irPR) | Decrease of ≥30% in TMTB relative to baseline, non-target |
| | lesions are irNN, and no unequivocal progression of new |
| | nonmeasurable lesions |
| | If new measurable lesions appear in subjects with no |
| | target lesions at baseline, irPD will be assessed. That |
| | irPD time point will be considered a new baseline, and |
| | all subsequent time points will be compared to it for |
| | response assessment. irPR is possible if the TMTB of |
| | new measurable lesions decreases by ≥30% compared |
| | to the first irPD documentation |
| | irRECIST can be used in the adjuvant setting, in |
| | subjects with no visible disease on CT/MRI scans. The |
| | appearance of new measurable lesion(s) automatically |
| | leads to an increase in TMTB by 100% and leads to |
| | irPD. These subjects can achieve a response if the |
| | TMTB decreases at follow-up, as a sign of delayed |
| | response. |
| | Based on the above, sponsors may consider enrolling |
| | subjects with no measurable disease and/or no visible |
| | disease in studies with response related endpoints. |
| Stable Disease (irSD) | Failure to meet criteria for irCR or irPR in the absence of irPD |
| Progressive Disease (irPD) | Minimum 20% increase and minimum 5 mm absolute increase |
| | in TMTB compared to nadir, or irPD for non-target or new |
| | non-measurable lesions. Confirmation of progression is |
| | recommended minimum 4 weeks after the first irPD |
| | assessment. An irPD confirmation scan may be recommended |
| | for subjects with a minimal TMTB %-increase over 20% and |
| | especially during the flare time-window of the first 12 weeks of |
| | treatment, depending on the compound efficacy expectations, to |
| | account for expected delayed response. |
| | In irRECIST a substantial and unequivocal increase of |
| | non-target lesions is indicative of progression. |
| | IrPD may be assigned for a subject with multiple new |
| | non-measurable lesions if they are considered to be a |
| | sign of unequivocal massive worsening |
| Other | irNE: used in exceptional cases where insufficient data exist. |
| | irND: in adjuvant setting when no disease is detected |
| | irNN:, no target disease was identified at baseline, and at |
| | follow-up the subject fails to meet criteria for irCR or irPD |

As used herein, a “sample” or “biological sample” refers to a body fluid or a tissue sample isolated from a subject. In some cases, a biological sample may consist of or comprise whole blood, platelets, red blood cells, white blood cells, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, endothelial cells, synovial fluid, lymphatic fluid, ascites fluid, interstitial or extracellular fluid and the like. The term “sample” may also encompass the fluid in spaces between cells, including gingival crevicular fluid, bone marrow, cerebrospinal fluid (CSF), saliva, mucus, sputum, semen, sweat, urine, or any other bodily fluids. Samples can be obtained from a subject by any means including, but not limited to, venipuncture, excretion, ejaculation, massage, biopsy, needle aspirate, lavage, scraping, surgical incision, or intervention or other means known in the art. A blood sample can be whole blood or any fraction thereof, including blood cells (red blood cells, white blood cells or leukocytes, and platelets), serum and plasma.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the terms “subject,” “individual,” or “patient” are used interchangeably and refer to an individual organism, a vertebrate, a mammal, or a human. In certain embodiments, the individual, patient or subject is a human.

“Treating”, “treat”, or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment or prevention of medical diseases and conditions as described are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

### Methotrexate

Methotrexate (MTX)-based regimens are the standard treatment for CNSL. MTX penetrates the blood brain barrier when administered at high doses (>1.5 g/m²) as a rapid infusion (Borsi, J. D. and P. J. Moe, Cancer 1987. 60(1): p. 5-13 (1987); Shapiro, W. R., D. F. Young, and B. M. Mehta, N Engl J Med, 293(4): p. 161-6 (1975)). A retrospective analysis of 357 patients suggested a statistically significant survival benefit with regimens that include doses 3 g/m² (Reni, M., et al., Int J Radiat Oncol Biol Phys, 2001. 51(2): p. 419-25 (2001)) though doses up to 8 g/m² are common in lymphoma management (Herrlinger, U., et al., Ann Neurol, 51(2): p. 247-52 (2002); Illerhaus, G., et al., J Clin Oncol, 24(24): p. 3865-70 (2006); Rubenstein, J. L., et al., J Clin Oncol, 31(25): p. 3061-8 (2013); Illerhaus, G., et al., Lancet Haematol, 3(8): p. e388-97 (2016)). The structure of MTX is provided below:

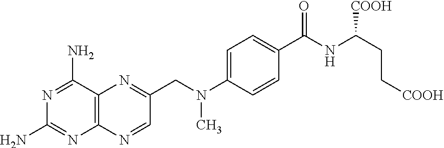 [SMILES: CN(Cc1cnc2nc(N)nc(N)c2n1)c1ccc(C(=O)N[C@@H](CCC(=O)O)C(=O)O)cc1]

While MTX is generally well tolerated, patients with MTX toxicity, renal failure, and other side effects may require dose reduction and rarely, cessation of therapy. MTX is predominantly cleared via renal excretion (70-90%) with contribution from the hepatic system via conversion of MTX to 7-hydroxymethotrexate. Between 2-12% of adults can sustain acute kidney injury as a result of HD-MTX. See Widemann, B. C., et al., J Clin Oncol, 28(25): p. 3979-86 (2010). MTX toxicity can sometimes be severe and fatal. MTX is a weak acid and most soluble at a lower pH. Precipitation of MTX in the renal tubules is a likely mechanism of nephrotoxicity (Garneau, A. P., J. Riopel, and P. Isenring, N Engl J Med, 373(27): p. 2691-3 (2015)) though vasoconstriction and direct tubular toxicity may also play a role. To reduce the risk of nephrotoxicity, supportive care is pre-emptively provided to all patients in the form of hydration (volume expansion) and urinary alkalinization. As a result, HD-MTX is almost always administered in the inpatient setting with continuous intravenous hydration and frequent monitoring of urine pH and serum MTX levels. Patients are typically maintained on supportive hydration with regular monitoring until serum levels reach ≤100 nmol, as measured by immunoassay. The half-life of IV MTX ranges between 8 and 12 hours and there is great inter- and intra-patient variability in clearance times. Treatments necessitate numerous hospitalizations of at least several days.

As dosage and administration of MTX is limited by systemic toxicity, rescue therapies for this have been developed. Leucovorin, or folinic acid, is a rescue agent administered to protect against the toxic systemic effects of MTX, and is metabolized to 5-methyl-tetrahydrofolate, providing a source of reduced folates able to bypass the effects of MTX. Leucovorin is dosed beginning 24 to 36 hours after MTX administration. Delays longer than 48 hours post-MTX are associated with increased risk of severe toxicity (Bertino, J. R., Semin Oncol, 4(2): p. 203-16 (1977)). Leucovorin competes with MTX for cellular uptake and for polyglutamylation intracellularly which enhances intracellular retention and affinity for target enzymes. Since it works by competitive inhibition, leucovorin is less effective when MTX levels are high. While leucovorin reduces the rate of toxic and fatal side effects of MTX, it does not eliminate the need for inpatient admission and close monitoring after MTX administration.

### Glucarpidase

Glucarpidase or carboxypeptidase G2 (e.g., Voraxaze®) is a recombinant bacterial enzyme that cleaves serum MTX to inactive metabolites. The amino acid sequence of glucarpidase is provided below:

> Glucarpidase
(SEQ ID NO: 1)
ALAQKRDNVLFQAATDEQPAVIKTLEKLVN
IETGTGDAEGIAAAGNFLEAELKNLGFTVT
RSKSAGLVVGDNIVGKIKGRGGKNLLLMSH
MDTVYLKGILAKAPFRVEGDKAYGPGIADD
KGGNAVILHTLKLLKEYGVRDYGTITVLFN
TDEEKGSFGSRDLIQEEAKLADYVLSFEPT
SAGDEKLSLGTSGIAYVQVNITGKASHAGA
APELGVNALVEASDLVLRTMNIDDKAKNLR
FNWTIAKAGNVSNIIPASATLNADVRYARN
EDFDAAMKTLEERAQQKKLPEADVKVIVTR
GRPAFNAGEGGKKLVDKAVAYYKEAGGTLG
VEERTGGGTDAAYAALSGKPVIESLGLPGF
GYHSDKAEYVDISAIPRRLYMAARLIMDLG
AGK

Voraxaze® is currently approved by the Food and Drug Administration (FDA) for patients with renal failure and MTX toxicity. The approved dose is 50 units/kg which has been demonstrated to be safe and efficacious in patients experiencing delayed MTX elimination as a result of renal impairment. At this dose, routine use of glucarpidase is cost-prohibitive. It is well-tolerated with fewer than 3% of patients reporting nausea, vomiting, hypotension, paresthesias, flushing, and headache. When MTX is cleaved, metabolites glutamate and 4-deoxy-4-amino-N10-methylpteroic acid (DAMPA) are formed. DAMPA is not cytotoxic and is eliminated primarily by the liver though data in non-human primates suggests urinary excretion also plays a role in clearance.

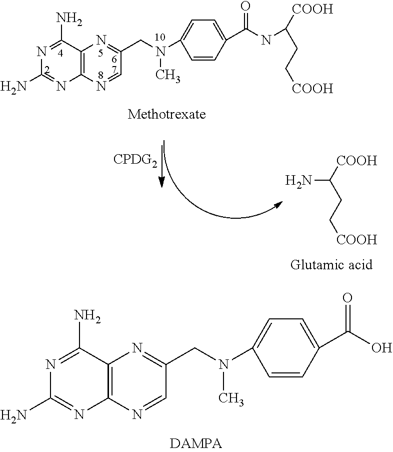 [SMILES: CN(Cc1cnc2nc(N)nc(N)c2n1)c1ccc(C(=O)NC(CCC(=O)O)C(=O)O)cc1 ; NC(CCC(=O)O)C(=O)O ; CN(Cc1cnc2nc(N)nc(N)c2n1)c1ccc(C(=O)O)cc1]

Glucarpidase is derived from a bacterial source (Pseudomonas sp. (strain RS-16)) and is potentially immunogenic. The development of antibodies has been described in roughly 17% of patients who received first or second doses of glucarpidase (Ramsey, L. B., et al., Oncologist 23(1): p. 52-61 (2018)). While the clinical significance of antibody development is not clear, there is the possibility that the formation of anti-glucarpidase antibodies would result in reduced glucarpidase efficacy for MTX cleavage and treatment of MTX toxicity. For example, EMEA Pre-authorisation Evaluation EMEA/CHMP/171907/2008 for use of glucarpidase in treating methotrexate toxicity noted that the antibody formation post glucarpidase was 37-43% in clinical studies and did not seem to be related to age or gender. In vitro data indicated that the antibodies may have some neutralising potency: serum from four of 22 patients with an antibody response inhibited glucarpidase enzymatic activity in vitro by 28-58%. As the anticipated use of glucarpidase in this scenario was one single dose, antibody formation was not considered a critical issue. However, the impact of the anti-glucarpidase antibodies on the efficacy of repeated administrations of low-dose glucarpidase for the purpose of MTX clearance is unknown.

Currently, Voraxaze® is not indicated for patients with expected clearance of MTX or with normal or only mildly impaired renal function because of concerns that glucarpidase administration would result in subtherapeutic doses of MTX, thus reducing the efficacy of CNSL therapy. Glucarpidase is a large molecule at 83 kDa and has a volume of distribution of 3.6 L, comparable to plasma. The half-life of glucarpidase is between 6 and 9 hours, independent of renal function. Glucarpidase is not believed to cross the blood brain barrier or cellular membranes due to its size though this has not been definitively demonstrated given the paucity of clinical data.

Formulations Including Methotrexate and/or Glucarpidase

The pharmaceutical compositions of the present technology can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain solvents, diluents, and other liquid vehicles, dispersion or suspension aids, surface active agents, pH modifiers, isotonic agents, thickening or emulsifying agents, stabilizers and preservatives, solid binders, lubricants and the like, as suited to the particular dosage form desired. In certain embodiments, the compositions disclosed herein are formulated for administration to a mammal, such as a human.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, cyclodextrins, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Compositions formulated for parenteral administration may be injected by bolus injection or by timed push, or may be administered by continuous infusion.

In order to prolong the effect of a compound(s) of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

The active compounds can also be in micro-encapsulated form with one or more excipients as noted above. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

### Modes of Administration and Effective Dosages

An effective amount of MTX or glucarpidase useful in the methods disclosed herein may be administered to a mammal in need thereof by any of a number of well-known methods for administering pharmaceutical compounds. The effective amount may be determined during pre-clinical trials and clinical trials by methods familiar to physicians and clinicians. MTX or glucarpidase may be administered systemically or locally.

MTX or glucarpidase can be incorporated into pharmaceutical compositions for administration, singly or in combination, to a subject for the treatment or prevention of a disease or condition disclosed herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The pharmaceutical compositions having MTX or glucarpidase can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic agent can be formulated in a carrier system. The carrier can be a colloidal system. The colloidal system can be a liposome, a phospholipid bilayer vehicle. In one embodiment, the therapeutic agent is encapsulated in a liposome while maintaining the agent's structural integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic agent can be embedded in the polymer matrix, while maintaining the agent's structural integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro.

### Therapeutic Methods

The following discussion is presented by way of example only, and is not intended to be limiting.

In one aspect, the present disclosure provides a method for treating central nervous system lymphoma in a subject in need thereof comprising (a) administering to the subject an effective amount of methotrexate; and (b) administering to the subject an amount of glucarpidase that is sufficient to reduce the level of methotrexate in the subject's serum by >90%, wherein the glucarpidase is administered 10 to 48 hours following administration of methotrexate, and wherein steps (a)-(b) are performed for 1, 2, 3, 4, 5, 6, 7, 8 or more cycles. In another aspect, the present disclosure provides a method for reducing the toxicity associated with high dose methotrexate therapy in a subject in need thereof comprising (a) administering to the subject an effective amount of methotrexate; and (b) administering to the subject an amount of glucarpidase that is sufficient to reduce the level of methotrexate in the subject's serum by >90%, wherein the glucarpidase is administered 10 to 48 hours following administration of methotrexate, wherein steps (a)-(b) are performed for 1, 2, 3, 4, 5, 6, 7, 8 or more cycles, and wherein the subject suffers from or is diagnosed with central nervous system lymphoma. The central nervous system lymphoma may be primary CNSL (PCNSL) or secondary CNSL (SCNSL). In a further embodiment, the PCNSL has developed in one or more tissues selected from the group consisting of brain parenchyma, spinal cord, meninges, cerebrospinal fluid, and eyes.

Additionally or alternatively, in some embodiments of the methods disclosed herein, the effective amount of methotrexate is about 3 to 10 g/m². In certain embodiments, the effective amount of methotrexate is about 3 g/m², about 3.5 g/m², 4 g/m², about 4.5 g/m², 5 g/m², about 5.5 g/m², 6 g/m², about 6.5 g/m², 7 g/m², about 7.5 g/m², 8 g/m², about 8.5 g/m², about 9 g/m², about 9.5 g/m², or about 10 g/m².

In any and all embodiments of the methods disclosed herein, the subject is suffering from or is diagnosed with diffuse large B-cell lymphoma (DLBCL). In certain embodiments, the subject exhibits metastases in one or more tissue sites selected from the group consisting of bone marrow, testicles, paranasal sinuses, bone, retroperitoneal lymph nodes and epidural space. Additionally or alternatively, in some embodiments, the subject is suffering from CNSL relapse.

In any of the preceding embodiments of the methods disclosed herein, glucarpidase and methotrexate are administered sequentially, or separately. In certain embodiments of the methods disclosed herein, methotrexate is administered subcutaneously, intravenously, intraperitoneally, intra-articularly, intra-synovially, intrasternally, intrathecally, orally, topically, transmucosally, iontophoretically, or via intracranial injection. Additionally or alternatively, in some embodiments, glucarpidase is administered subcutaneously, intravenously, intraperitoneally, intra-articularly, intra-synovially, intrasternally, intrathecally, orally, topically, transmucosally, iontophoretically, or via intracranial injection. In some embodiments, the subject is human.

Additionally or alternatively, in some embodiments, steps (a)-(b) are performed for up to 10 cycles. In other embodiments, steps (a)-(b) are performed for 3-8 cycles. In any and all embodiments of the methods disclosed herein, the amount of glucarpidase in at least one cycle is about 1800 to 2200 units. Additionally or alternatively, in some embodiments, the amount of glucarpidase in at least one cycle is about 800 to 1200 units. In certain embodiments, the amount of glucarpidase in at least one cycle is about 800 units, about 850 units, about 900 units, about 950 units, about 1000 units, about 1050 units, about 1100 units, about 1150 units, about 1200 units, about 1250 units, about 1300 units, about 1350 units, about 1400 units, about 1450 units, about 1500 units, about 1550 units, about 1600 units, about 1650 units, about 1700 units, about 1750 units, about 1800 units, about 1850 units, about 1900 units, about 1950 units, about 2000 units, about 2050 units, about 2100 units, about 2150 units, or about 2200 units. In any of the preceding embodiments of the methods disclosed herein, the amount of glucarpidase in the first 2 to 4 cycles is about 1800 to 2200 units and the amount of glucarpidase after the first 2 to 4 cycles is about 800 to 1200 units. In certain embodiments, the amount of glucarpidase in the first 2 to 4 cycles is about 2000 units and the amount of glucarpidase after the first 2 to 4 cycles is about 1000 units.

Additionally or alternatively, in some embodiments, the methods of the present technology further comprise administering to the subject an effective amount of an additional therapeutic agent that targets CNSL. The additional therapeutic agent that targets CNSL may be an anti-CD20 antibody, an anti-CD19 antibody, a steroid (e.g., glucocorticoids), a chemotherapeutic agent, and any combination thereof. Examples of anti-CD20 antibodies include, but are not limited to rituximab (e.g., MabThera®, Rixathon® and Truxima®), ocrelizumab, obinutuzumab, veltuzumab, ofatumumab, ibritumomab tiuxetan, ¹³¹I tositumomab, AME-133v, PRO131921, TRU-015, and GA101. Examples of anti-CD19 antibodies include, but are not limited to Blinatumomab, GBR 401, Coltuximabravtansine, MOR208, MEDI-551, Denintuzumabmafodotin, Taplitumomabpaptox, XmAb 5871, MDX-1342, AFM11, and SAR3419 (huB4-DM4). Additionally or alternatively, in some embodiments, the at least one additional therapeutic agent is a chemotherapeutic agent selected from the group consisting of cyclophosphamide, carmustine, etoposide, bisulfan, vincristine, procarbazine, temozolomide, cytarabine, and thiotepa.

In any of the preceding embodiments of the methods disclosed herein, the anti-CD20 antibody is administered in one or more cycles with methotrexate and glucarpidase. In certain embodiments, each cycle occurs over (i) a three day period and (ii) comprises: administering to the subject about 300-600 mg/m² of anti-CD20 antibody on day 1, administering to the subject about 3-10 mg/m² methotrexate on day 2, and administering to the subject about 800-2200 units of glucarpidase on day three. In certain embodiments, the amount of anti-CD20 antibody administered to the subject on day 1 is about 300 mg/m², about 350 mg/m², about 400 mg/m², about 450 mg/m², about 500 mg/m², about 550 mg/m², or about 600 mg/m².

In any and all embodiments of the methods disclosed herein, the subject exhibits a delay in metastatic onset and/or tumor growth after administration of MTX and glucarpidase compared to that observed in an untreated control subject diagnosed with CNSL.

Additionally or alternatively, in some embodiments of the methods disclosed herein, MTX and glucarpidase are administered sequentially, simultaneously, or separately. MTX and/or glucarpidase may be administered orally, parenterally, by inhalation spray, intranasally, buccally, or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intravenously, or subcutaneously. Formulations including MTX and/or Glucarpidase may be designed to be short-acting, fast-releasing, or long-acting. In other embodiments, compounds can be administered in a local rather than systemic means, such as administration (e.g., by injection) at a tumor site.

Additionally or alternatively, in some embodiments of the methods disclosed herein, MTX can be administered prior to (e.g., 5 minutes, 15 minutes, 30 minutes, 45 minutes, 1 hour, 2 hours, 4 hours, 6 hours, 8 hours, 10 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), simultaneously with, or subsequent to (e.g., 5 minutes, 15 minutes, 30 minutes, 45 minutes, 1 hour, 2 hours, 4 hours, 6 hours, 8 hours, 10 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of glucarpidase to a patient with CNSL.

In some embodiments, MTX and glucarpidase are administered to a patient, for example, a mammal, such as a human, in a sequence and within a time interval such that the two compositions act together to provide a greater benefit than if each composition were administered alone. For example, MTX and glucarpidase can be administered at the same time or sequentially in any order at different points in time; however, if not administered at the same time, MTX and glucarpidase are administered sufficiently close in time so as to provide the desired therapeutic or prophylactic effect of the combination of the two compositions. In one embodiment, MTX and glucarpidase exert their effects at times which overlap. In some embodiments, MTX and glucarpidase are each administered as separate dosage forms, in any appropriate form and by any suitable route. In other embodiments, MTX and glucarpidase are administered simultaneously in a single dosage form.

It will be appreciated that the frequency with which any of these therapeutic agents can be administered can be once or more than once over a period of about 2 days, about 3 days, about 4 days, about 5 days, about 6 days, about 7 days, about 8 days, about 9 days, about 10 days, about 11 days, about 12 days, about 13 days, about 14 days, about 20 days, about 24 days, about 28 days, about a week, about 2 weeks, about 3 weeks, about 4 weeks, about a month, about every 2 months, about every 3 months, about every 4 months, about every 5 months, about every 6 months, about every 7 months, about every 8 months, about every 9 months, about every 10 months, about every 11 months, about every year, about every 2 years, about every 3 years, about every 4 years, or about every 5 years.

For example, the MTX and glucarpidase regimen (MTX/glucarpidase regimen) may be administered weekly, biweekly (after every 2 weeks), or monthly for 1, 2, 3, 4, 5, 6, 7, 8 or more cycles. Additionally or alternatively, in some embodiments, each cycle is a 14-day cycle, a 13-day cycle, a 12-day cycle, an 11-day cycle, a 10-day cycle, a 9-day cycle, an 8-day cycle, a 7-day cycle, a 6-day cycle, a 5-day cycle, a 4-day cycle, or a 3-day cycle.

Additionally or alternatively, in some embodiments, the MTX/glucarpidase regimen may be administered once a week followed by a particular period of non-treatment, or twice a week wherein the first dose of the MTX/glucarpidase regimen is followed by a particular period of non-treatment (e.g., 1, 2, 3, 4, or 5 days) prior to administration of the second dose of the MTX/glucarpidase regimen. Additionally or alternatively, in some embodiments, the MTX and glucarpidase can be sequentially administered on the same day, followed by 6-27 days of non-treatment. In other embodiments, MTX is administered on day 1, glucarpidase is administered on any of day 2, day 3, day 4, or day 5, followed by 5-26 days of non-treatment.

In some embodiments, individual doses of MTX and glucarpidase are administered within a time interval such that the two compositions can work together (e.g., within 1 hour, 2 hours, 4 hours, 6 hours, 12 hours, 24 hours, 48 hours, 72 hours, 96 hours, 5 days, 6 days, 1 week, or 2 weeks). In some embodiments, the treatment period during which the therapeutic agents are administered is then followed by a non-treatment period of a particular time duration, during which the therapeutic agents are not administered to the patient. This non-treatment period can then be followed by a series of subsequent treatment and non-treatment periods of the same or different frequencies for the same or different lengths of time. In some embodiments, the treatment and non-treatment periods are alternated. It will be understood that the period of treatment in cycling therapy may continue until the patient has achieved a complete response or a partial response, at which point the treatment may be stopped. Alternatively, the period of treatment in cycling therapy may continue until the patient has achieved a complete response or a partial response, at which point the period of treatment may continue for a particular number of cycles. In some embodiments, the length of the period of treatment may be a particular number of cycles, regardless of patient response. In some other embodiments, the length of the period of treatment may continue until the patient relapses.

In some embodiments, MTX and glucarpidase are cyclically administered to a patient. Cycling therapy involves the administration of a first agent (e.g., a first prophylactic or therapeutic agent) for a period of time, followed by the administration of a second agent and/or third agent (e.g., a second and/or third prophylactic or therapeutic agent) for a period of time and repeating this sequential administration. Cycling therapy can reduce the development of resistance to one or more of the therapies, avoid or reduce the side effects of one of the therapies, and/or improve the efficacy of the treatment.

In some embodiments, MTX is administered for a particular length of time prior to administration of glucarpidase. For example, in a 21-day cycle, MTX may be administered on days 1 to 5, days 1 to 7, days 1 to 10, or days 1 to 14, and glucarpidase may be administered on days 6 to 21, days 8 to 21, days 11 to 21, or days 15 to 21. In other embodiments, glucarpidase is administered for a particular length of time prior to administration of MTX. For example, in a 21-day cycle, glucarpidase may be administered on days 1 to 5, days 1 to 7, days 1 to 10, or days 1 to 14, and MTX may be administered on days 6 to 21, days 8 to 21, days 11 to 21, or days 15 to 21.

In one embodiment, the administration is on a dose schedule in which MTX is administered beginning on day 1, followed by glucarpidase about 4-96 hours (e.g., 4 hours, 6 hours, 8 hours, 10 hours, 12 hours, 24 hours, 48 hours, 72 hours, 96 hours,) after MTX administration (e.g., glucarpidase is administered on day 1, 2, 3, 4, or 5 and MTX is administered on day 1 of the schedule), followed by 0-28 days of non-treatment.

In some embodiments, MTX and glucarpidase are each administered at a dose and schedule typically used for that agent during monotherapy. In other embodiments, when MTX and glucarpidase are administered concomitantly, one or both of the agents can advantageously be administered at a lower dose than typically administered when the agent is used during monotherapy, such that the dose falls below the threshold that an adverse side effect is elicited.

The therapeutically effective amounts or suitable dosages of MTX and glucarpidase in combination depends upon a number of factors, including the nature of the severity of the condition to be treated, the particular therapeutic composition, the route of administration and the age, weight, general health, and response of the individual patient. In certain embodiments, the suitable dose level is one that achieves a therapeutic response as measured by lymphoma regression or other standard measures of disease progression, progression free survival, or overall survival. In other embodiments, the suitable dose level is one that achieves this therapeutic response and also minimizes any side effects associated with the administration of the therapeutic agent.

Suitable daily dosages of MTX can generally range, in single or divided or multiple doses, from about 10% to about 120% of its maximum tolerated dose. In certain embodiments, the suitable dosages of MTX are from about 20% to about 100% of its maximum tolerated dose. In other embodiments, the suitable dosages of MTX are from about 25% to about 90% of its maximum tolerated dose. In some embodiments, the suitable dosages of MTX are from about 30% to about 80% of its maximum tolerated dose. In other embodiments, the suitable dosages of MTX are from about 40% to about 75% of its maximum tolerated dose. In some embodiments, the suitable dosages of MTX are from about 45% to about 60% of its maximum tolerated dose. In other embodiments, suitable dosages of MTX are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of its maximum tolerated dose.

Suitable daily dosages of glucarpidase can generally range, in single or divided or multiple doses, from about 10% to about 120% of its maximum tolerated dose. In certain embodiments, the suitable dosages of glucarpidase are from about 20% to about 100% of its maximum tolerated dose. In some other embodiments, the suitable dosages of glucarpidase are from about 25% to about 90% of its maximum tolerated dose. In some other embodiments, the suitable dosages of glucarpidase are from about 30% to about 80% of its maximum tolerated dose. In some other embodiments, the suitable dosages of glucarpidase are from about 40% to about 75% of its maximum tolerated dose. In some other embodiments, the suitable dosages of glucarpidase are from about 45% to about 60% of its maximum tolerated dose. In other embodiments, suitable dosages of glucarpidase are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of its maximum tolerated dose.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of MTX or glucarpidase, sufficient for achieving a therapeutic or prophylactic effect, may range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of MTX or glucarpidase ranges from 0.001-10,000 micrograms per kg body weight. In one embodiment, MTX or glucarpidase concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of a MTX or glucarpidase may be defined as a concentration of MTX or glucarpidase at the target tissue of 10⁻¹² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

Combination Therapy with Other Therapeutic Agents Targeting CNS Lymphomas

In some embodiments, one or more of MTX or glucarpidase may be combined with one or more additional therapies for the prevention or treatment of CNSL. Additional therapeutic agents include, but are not limited to, anti-CD20 antibodies, anti-CD19 antibodies, steroid therapy (e.g., glucocorticoids), chemotherapeutic agents, High-dose chemotherapy with stem cell transplant, radiation therapy, or any combination thereof.

In some embodiments, MTX and/or glucarpidase may be separately, sequentially or simultaneously administered with at least one additional therapeutic agent selected from the group consisting of anti-CD20 antibodies, anti-CD19 antibodies, steroid therapy (e.g., glucocorticoids), chemotherapeutic agents, High-dose chemotherapy with stem cell transplant, radiation therapy, or any combination thereof. Examples of anti-CD20 antibodies include, but are not limited to rituximab (e.g., MabThera®, Rixathon® and Truxima®), ocrelizumab, obinutuzumab, veltuzumab, ofatumumab, ibritumomab tiuxetan, ¹³¹I tositumomab, AME-133v, PRO131921, TRU-015, and GA101. Examples of anti-CD19 antibodies include, but are not limited to Blinatumomab, GBR 401, Coltuximabravtansine, MOR208, MEDI-551, Denintuzumabmafodotin, Taplitumomabpaptox, XmAb 5871, MDX-1342, AFM11, and SAR3419 (huB4-DM4). See Naddafi et al., Int J Mol Cell Med. 4(3): 143-151 (2015). Additionally or alternatively, in some embodiments, MTX and/or glucarpidase may be separately, sequentially or simultaneously administered with at least one additional therapeutic agent selected from the group consisting of cyclophosphamide, carmustine, etoposide, bisulfan, vincristine, procarbazine, temozolomide, cytarabine, and thiotepa.

Additionally or alternatively, in some embodiments, MTX and/or glucarpidase may be separately, sequentially or simultaneously administered with at least one additional therapeutic agent. Examples of the at least one additional therapeutic agent include, but are not limited to alkylating agents, topoisomerase inhibitors, endoplasmic reticulum stress inducing agents, antimetabolites, mitotic inhibitors, nitrogen mustards, nitrosoureas, alkylsulfonates, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents, phenphormin and targeted biological therapy agents (e.g., therapeutic peptides described in U.S. Pat. No. 6,306,832, WO 2012007137, WO 2005000889, WO 2010096603 etc.). In some embodiments, the at least one additional therapeutic agent is a chemotherapeutic agent.

Specific chemotherapeutic agents include, but are not limited to, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), cladribine, midostaurin, bevacizumab, oxaliplatin, melphalan, etoposide, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, epirubicin, idarubicin, SN-38, ARC, NPC, campothecin, 9-nitrocamptothecin, 9-aminocamptothecin, rubifen, gimatecan, diflomotecan, BN80927, DX-8951f, MAG-CPT, amsacnne, etoposide phosphate, teniposide, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, camptothecin, exatecan, lurtotecan, lamellarin D9-aminocamptothecin, amsacrine, ellipticines, aurintricarboxylic acid, HU-331, or combinations thereof.

Examples of antimetabolites include 5-fluorouracil (5-FU), 6-mercaptopurine (6-MP), capecitabine, cytarabine, floxuridine, fludarabine, gemcitabine, hydroxyurea, methotrexate, pemetrexed, and mixtures thereof.

Examples of taxanes include accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, and mixtures thereof.

Examples of DNA alkylating agents include cyclophosphamide, chlorambucil, melphalan, bendamustine, uramustine, estramustine, carmustine, lomustine, nimustine, ranimustine, streptozotocin; busulfan, mannosulfan, and mixtures thereof.

Examples of topoisomerase I inhibitor include SN-38, ARC, NPC, camptothecin, topotecan, 9-nitrocamptothecin, exatecan, lurtotecan, lamellarin D9-aminocamptothecin, rubifen, gimatecan, diflomotecan, BN80927, DX-8951f, MAG-CPT, and mixtures thereof. Examples of topoisomerase II inhibitors include amsacrine, etoposide, etoposide phosphate, teniposide, daunorubicin, mitoxantrone, amsacrine, ellipticines, aurintricarboxylic acid, doxorubicin, and HU-331 and combinations thereof.

In any case, the multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

### Kits

The present disclosure provides kits comprising MTX, glucarpidase, and instructions for treating CNSL in accordance with any and all embodiments of the methods disclosed herein. When simultaneous administration is contemplated, the kit may comprise MTX and glucarpidase that has been formulated into a single pharmaceutical composition such as a tablet, or as separate pharmaceutical compositions. When MTX and glucarpidase are not administered simultaneously, the kit may comprise MTX and glucarpidase that has been formulated as separate pharmaceutical compositions either in a single package, or in separate packages. Optionally, the above described components of the kits of the present technology are packed in suitable containers and labeled for the treatment of CNSL. The CNSL may be primary CNSL (PCNSL) or secondary CNSL (SCNSL).

Additionally or alternatively, in some embodiments, the kits further comprise at least one additional therapeutic agent disclosed herein that is useful for treating CNSL. Examples of such therapeutic agents include, but are not limited to steroids (e.g., glucocorticoids), rituximab, ocrelizumab, obinutuzumab, veltuzumab, ofatumumab, ibritumomab tiuxetan, ¹³¹I tositumomab, AME-133v, PRO131921, TRU-015, GA101, Blinatumomab, GBR 401, Coltuximabravtansine, MOR208, MEDI-551, Denintuzumabmafodotin, Taplitumomabpaptox, XmAb 5871, MDX-1342, AFM11, SAR3419 (huB4-DM4), cyclophosphamide, carmustine, etoposide, bisulfan, vincristine, procarbazine, temozolomide, cytarabine, and thiotepa.

The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. The kits may optionally include instructions customarily included in commercial packages of therapeutic products, that contain information about, for example, the indications, usage, dosage, manufacture, administration, contraindications and/or warnings concerning the use of such therapeutic products.

The kit can also comprise, e.g., a buffering agent, a preservative or a stabilizing agent. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit. In certain embodiments, the use of the reagents can be according to the methods of the present technology.

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: Combination Therapy Methods of the Present Technology are Useful for Treating CNS Lymphoma in Patients

Methods: Eligible patients having CNS Lymphoma without systemic involvement, KPS ≥50, and age ≥18 with normal end organ function were selected for treatment with rituximab and methotrexate by their treating oncologist. Patients were enrolled into two cohorts with Cohort A receiving MTX 3 g/m² and Cohort B receiving MTX 6 g/m². Glucarpidase 2000 U or 1000 U was administered 24 hours following the initiation of MTX infusion. A cycle is typically 14±7 days. In the present Example, rituximab was administered on Day 1, MTX on Day 2, glucarpidase Day 3, followed by an 11-day non-treatment period before the start of the next cycle.

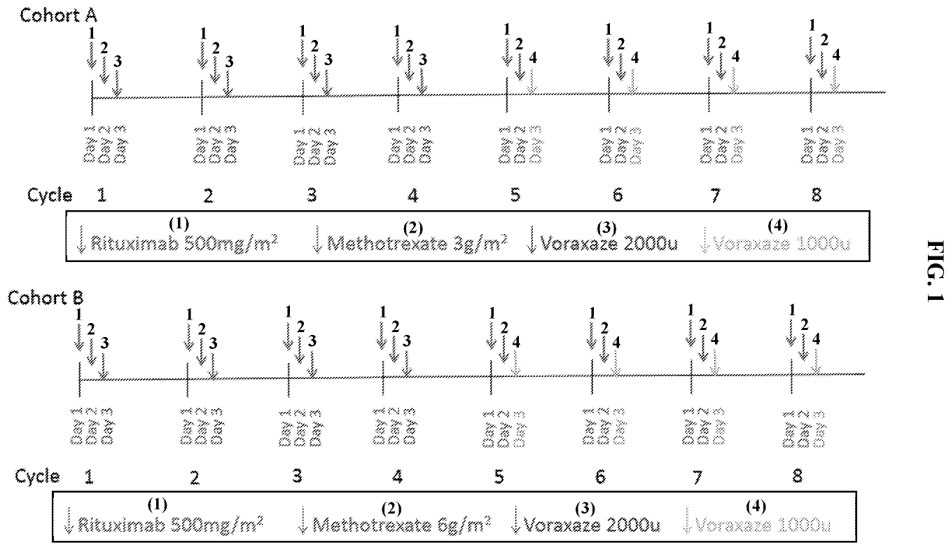

Serum was collected at pre-specified time points (e.g., pre-glucarpidase, 1 hour post glucarpidase administration, 6 hours post glucarpidase administration, 24 hours post glucarpidase administration, then every 24 hours until clearance) and concentrations of MTX and 2,4-diamino-N¹⁰-methylpteroic acid (DAMPA) were analyzed by mass spectrometry. Cerebrospinal fluid (CSF) was collected throughout treatment. Serum was collected for analysis of anti-glucarpidase antibodies. FIG. 1 illustrates the dosage regimen administered to subjects in Cohort A and Cohort B. Rituximab, MTX and glucarpidase were administered intravenously. Rituximab was always dosed at 500 mg/m².

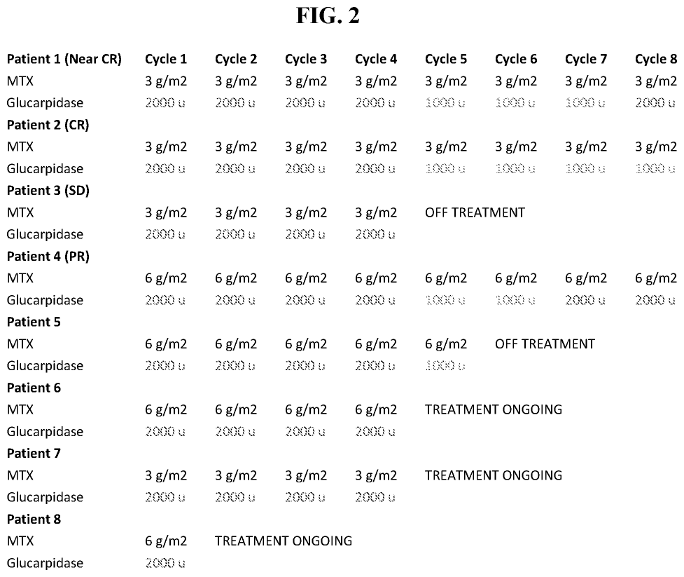

Results: 8 patients were enrolled in the study. FIG. 2 shows the treatment regimen for each of the 8 enrolled patients. A total of 42 doses of MTX were collectively administered to the 8 patients (24 doses at 3 g/m² and 24 doses at 6 g/m²). Patients collectively received 32 doses of glucarpidase 2000 U and 10 doses of glucarpidase 1000 U. See FIG. 2.

Out of the 5 enrolled patients, administration of glucarpidase resulted in at least a 95% reduction in serum MTX levels within 15 minutes of administration in 23/24 doses (96%). A 93% decrease was seen with the remaining dose (2000 U). In 23/24 doses, MTX levels were reduced to less than 100 nmol/L within 15 minutes of glucarpidase. A transient increase in MTX levels above 100 nmol/L was seen following 8 of 24 doses (33%), typically around 24 hours after administration of glucarpidase (median elevation of MTX to 290 nmol/L; range 119-516 nmol/L). CSF methotrexate concentrations were evaluated in two patients and remained therapeutic following glucarpidase administration.

End of treatment response was evaluated in two patients, one with complete response (CR) and one with near CR. Mid-treatment response was evaluated in two additional patients, one with partial response (PR) and one with stable disease (SD). One patient was not eligible for response assessment due to ongoing treatment. Three grade 3 adverse events were seen in two patients, which involved lung infection (pneumocystis pneumonia), sepsis related to central line infection, and decreased lymphocyte count. No grade 4 or 5 adverse events occurred. There were no adverse events associated with glucarpidase administration. The most common adverse events were hypoglycemia unlikely related to treatment (Holdhoff, M., et al., Neurology, 83(3): p. 235-9 (2014), anemia and decreased lymphocyte count (Kansara, R., et al., Am J Hematol, 90(12): p. 1149-54 (2015)). Analysis of anti-glucarpidase antibodies was to be determined at a later time point.

Administration of low-dose glucarpidase 24 hours after MTX resulted in rapid reduction in serum MTX levels. In patients analyzed to date, CSF MTX levels remained therapeutic and clinical response was observed. Anti-glucarpidase antibody titer levels were obtained for two patients. The first patient exhibited detectable anti-glucarpidase antibodies, but was still responsive to the repeated doses of the MTX/glucarpidase regimen. The second patient did not develop any anti-glucarpidase antibodies and responded well to the MTX/glucarpidase regimen.

These results demonstrate that the combination therapy methods of the present technology are useful in methods for treating CNS Lymphoma in a subject in need thereof.

### Example 2: Extended Effects of Combination Therapy Methods of the Present Technology in CNS Lymphoma Patients

Methods. In a phase I dose-finding study of planned-use glucarpidase following HD-MTX, patients were treated with either MTX at 3 g/m², 6 g/m², or 8 g/m². Glucarpidase was dose-reduced to 1000 U or 2000 U and given 24 hours after MTX. To date, 50 treatments have been administered across 12 patients. MTX levels following glucarpidase were determined via mass spectrometry as immunoassay fails to differentiate MTX from byproducts.

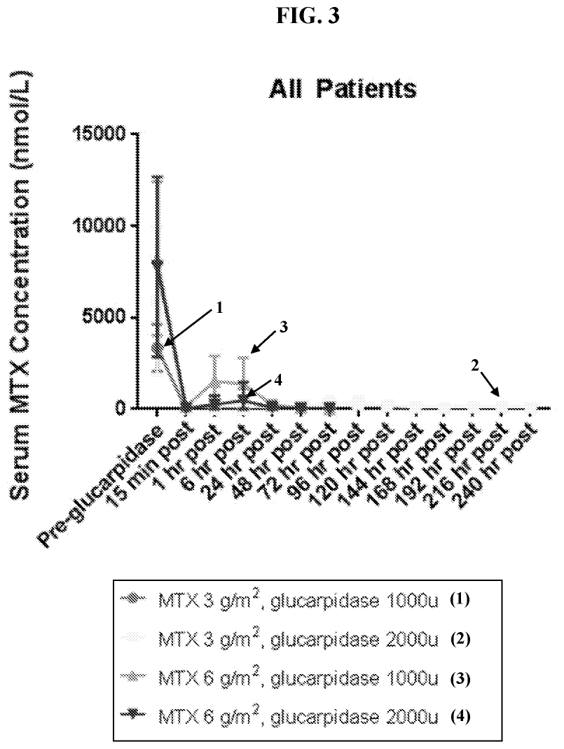

Glucarpidase 1000 U (14 treatments) or 2000 U (36 treatments) resulted in at least a 95% reduction in serum MTX levels within 15 minutes following 49/50 doses. A 93% decrease was seen with the remaining dose (FIG. 3). Rebound in MTX levels was seen after 19/50 doses across 5 patients, most frequently 6 hours after glucarpidase. Rebound occurred following 11/14 doses of glucarpidase 1000 U reaching an average of 39% of starting MTX levels and 9/36 doses of glucarpidase 2000 U reaching an average of 18% of starting MTX levels. Glucarpidase was not detected in the CSF of 7 patients analyzed to date. Anti-glucarpidase antibodies were detected in 6 of 8 patients analyzed to date.

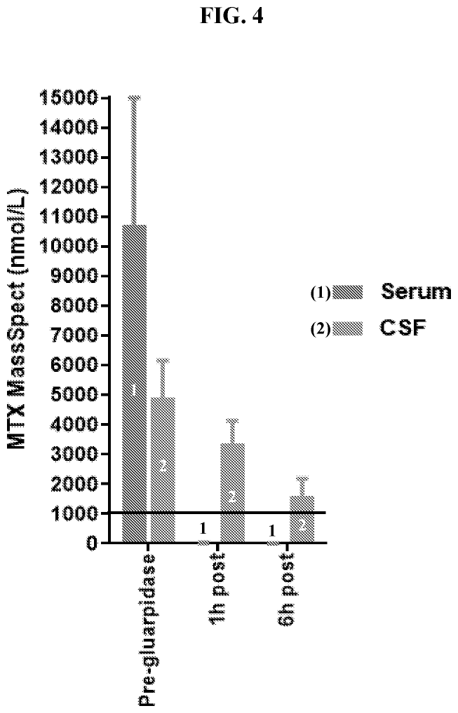

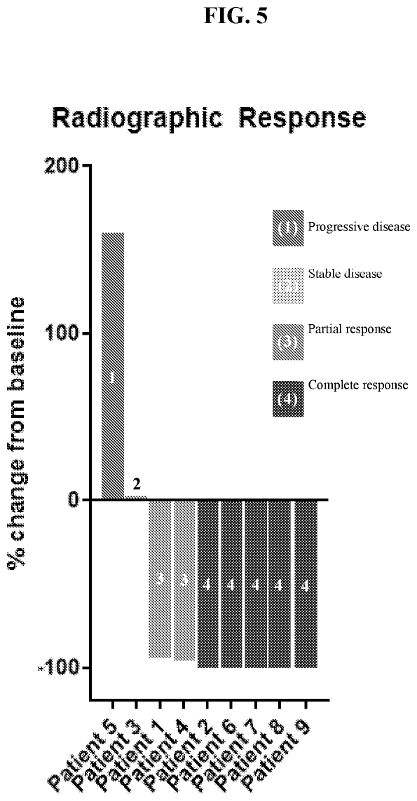

Potentially cytotoxic MTX concentrations (10⁻⁶ M) were observed in CSF 25 hours (10/11) following MTX administration (1 hour after glucarpidase) (FIG. 4). Radiographic responses are evaluable in 9 patients: 5 had a complete and 2 a partial response, with an overall response rate (ORR) of 78% (FIG. 5). One patient had stable disease, and one patient had progressive disease. The non-responding patient had multiple recurrences and failed three prior treatments. Glucarpidase has been well tolerated with only one grade 4 anaphylactic reaction and no grade 3 events. Based on these data, a dose of 2000 U was selected for future studies.

These results demonstrate that low-dose glucarpidase is effective in reducing MTX levels >95% and remains effective even after repeated doses and despite the formation of anti-glucarpidase antibodies. These results were unexpected given that previous studies have reported that such anti-glucarpidase antibodies can have neutralizing potency, and thus adversely impact glucarpidase enzymatic activity in patients receiving the MTX/glucarpidase therapeutic regimen. See Adamson et al., J. Clinical Oncology 10(8): 1359-1364 (1992); EMEA Pre-authorisation Evaluation EMEA/CHMP/171907/2008. In fact, glucarpidase was not detected in the CSF of patients, and CSF MTX concentrations following glucarpidase remain at cytotoxic levels. Clinical response remained high with a response rate of 78%.

Accordingly, these results demonstrate that the combination therapy methods of the present technology are useful in methods for treating CNS Lymphoma in a subject in need thereof.

### Example 3: Feasibility of Outpatient HD-MTX Administration in Combination with Planned-Use Low-Dose Glucarpidase in CNS Lymphoma Patients

In the spring of 2020, the COVID-19 pandemic was at its peak in New York City.

Elective procedures and admissions were canceled to create bed space for COVID-19 infected patients. To minimize face-to-face patient contact during HD-MTX treatment, the present study was amended to pilot the planned use of low-dose glucarpidase with outpatient HD-MTX administration. The study allowed for 10 single outpatient HD-MTX treatments. Eligible patients had normal renal function at baseline and had previously tolerated standard inpatient HD-MTX therapy. Patients were treated in outpatient chemotherapy infusion suites with MTX at 3.5 g/m², pre- and post-hydration on day 1. Patients returned the next day for additional IV hydration and glucarpidase 2000 U, then on day 3 for an MTX level by mass spectrometry. Triggers for hospitalization were defined by MTX levels and development of a grade ≥3 creatinine elevation. To date, 7 outpatient HD-MTX treatments have been administered to 3 patients. In all cases, serum MTX levels were reduced to <100 nmol/L at 48 hours. Hospital admission was not required after any treatment. Three treatments resulted in grade 1 AST/ALT elevation (2 patients). One treatment resulted in a grade 2 creatinine increase which resolved after additional outpatient hydration.

These results demonstrate the feasibility of outpatient HD-MTX administration in combination with planned-use low-dose glucarpidase.

Accordingly, these results demonstrate that the combination therapy methods of the present technology are useful in methods for treating CNS Lymphoma in a subject in need thereof.

### Example 4: Extended Analyses of Outpatient HD MTX Administration in Combination with Planned-Use Low-Dose Glucarpidase in CNS Lymphoma Patients

The objective of the present study is to determine the safety and tolerability of outpatient HD-MTX followed by glucarpidase (2000 units), and to characterize the rate of overall tumor response in CNSL patients treated with the same, as defined by the International Primary CNSL Collaborate Group (IPCG) guidelines.

Methods. 12 patients with newly diagnosed or relapsed CNSL that are eligible for treatment with HD-MTX at 3.5 g/m² will be recruited. Patients with active systemic disease are excluded because of the potential for glucarpidase to interfere with systemic therapy. Patients with baseline renal dysfunction are also excluded due to increased risk of MTX-related nephrotoxicity. All patients will be planned for 8 cycles of HD-MTX at a dose of 3.5 g/m². Additional agents such as rituximab, vincristine, and procarbazine may be administered concurrently at the discretion of the treating physician and in accordance with standard of care.

Treatment administration: On cycle day 1, patients will receive pre-hydration with D5W NaHCO₃ over 4 hours, followed by MTX at 3.5 g/m², then an additional 2 hours of hydration. Glucarpidase 2000 U is administered 24 hours after MTX on day 2, along with additional hydration. Blood for MTX level is drawn on day 3 and processed by mass spectrometry. The first two cycles will be administered inpatient to ensure tolerability, the following six will occur outpatient. This study allows for physician's choice for consolidation therapy following completion of MTX therapy.

Monitoring: Laboratory assessments including complete blood count, a comprehensive metabolic panel, and urine pH are performed on days 1-3. MTX levels are assessed on days 2 and 3. MTX levels drawn within 48 hours following glucarpidase administration are processed by mass spectrometry. During outpatient cycles (3-8) patients check and record urine pH at home until MTX clearance is documented. If urine pH falls <7.0, patients take additional oral sodium bicarbonate. Neuroimaging and CSF sampling will occur at baseline, after cycle 4, and at end of treatment. All patients will undergo assessment for adverse events which will be summarized based on the Common Toxicity Criteria for Adverse Events (CTCAE) version 5.0. AEs will be reported and described in terms of incidence and severity. Response will be determined based on the IPCG guidelines for tumor response assessment. Prospective studies report overall response rate (ORR) to MTX-based therapy in 50-90% of treated patients. Based on this experience and the small sample size of this study, it is anticipated that 70% of the enrolled patients will show a response to therapy.

CSF will be sampled in cycles 1 and 2, 6 hours and 1 hour following glucarpidase administration, respectively. CSF will be analyzed for MTX concentration and presence of glucarpidase. Collected data will be de-identified and stored in Medidata for analysis. CSF samples obtained throughout this study will be used to further correlate CSF MTX concentrations with response and survival metrics.

Patient-Reported Outcome (PRO) measures will be administered and semi-structured qualitative interviews will be performed with patients enrolled in the study of outpatient MTX in combination with glucarpidase. 12 additional patients will also be enrolled for standard of care HD-MTX in the inpatient setting to serve as contemporary controls. PROs will be assessed in all patients at baseline, mid-way through HD-MTX treatment (cycle 4), and at end of treatment. Patients will receive a battery of surveys to assess symptoms from CNSL, side effects related to treatment, psychological and emotional well-being during treatment, and time spent in contact with the health care system. The following metrics will be obtained

(1) The FACT-CNS (version 5) a 39-item compilation of general questions divided into five domains: Physical Well-Being, Social/Family Well-Being, Emotional Well-Being, Functional Well-Being, and a CNS subscale. It is a supplement to the FACT-G, a scale well-validated in the oncology population, and is appropriate for use in adult patients with CNS malignancy. This measure is being obtained to capture physical and existential symptoms of disease throughout the treatment course;

(2) PRO version of the CTCAE (PRO-CTCAE): developed by the National Cancer Institute to evaluate symptomatic toxicity in patients on cancer clinical trials and serves as a companion to the CTCAE standard scale for AE reporting. It is included to capture side effects/toxicity from treatment;

(3) Hospital Anxiety and Depression Scale, a brief assessment validated in the cancer population that indicates presence or absence of anxiety/depression and correlates with severity;

(4) the Temporal Aspects of Cancer Care survey is a 5 minute survey for use in the brain tumor population. It assesses quality of life and addresses how much patient time is spent in contact with the healthcare system. The total survey time is 35 minutes. Surveys will be administered during treatment. In the case of early termination of study participation, the end of treatment surveys will be obtained either in clinic or on the patient's own time.

To supplement the PRO data, semi-structured qualitative interviews will be conducted with both groups of patients. To understand patient experiences across the treatment trajectory, and assess change in perceptions over time, patients will participate in a total of three interviews: prior to treatment initiation, mid-way (cycle 4) and end of treatment. A separate semi-structured moderator's guide will be developed for each time point and will include flexibility for the moderator to ask follow-up questions (“probes”) to enable patients to elaborate. Interviews will attempt to capture impact of healthcare utilization and family burden, as well as elicit other themes. Interviews will last up to 30 minutes and will be audio-recorded and transcribed for analysis.

PRO Administration: PRO surveys will be administered to the 12 patients enrolled in the study for outpatient HD-MTX with glucarpidase. Additionally, consent will be obtained from 12 control patients with isolated CNSL who are receiving standard HD-MTX in the inpatient setting. Control patients will receive the same surveys at the same time points of treatment. After four interviews have been completed across all patients, a quality assurance review will be performed to evaluate concordance between interviewers.

Chart surveys and patient interviews will be conducted at cycle 4 (mid-way) and at the end of treatment to determine unplanned points of contact with the healthcare system since treatment initiation. These assessments will capture unplanned hospital admissions, the number of admission days, unplanned emergency room or urgent care visits, and unplanned outpatient clinic visits to medical specialists of any kind and primary care providers. The number of hours spent in contact with the healthcare system (inpatient and outpatient) based on visit and admission check-in and check-out times will be tabulated. When not available, estimated times will be provided by patients.

It is expected that outpatient HD-MTX in combination with glucarpidase is both safe and effective. These results will provide baseline information on PROs with standard CNSL treatment and standardize these metrics for future CNSL trials. In addition, it is anticipated that qualitative interviews will uncover new CNSL patient experience themes, which can be used to generate new items for future PROs

Accordingly, these results demonstrate that the combination therapy methods of the present technology are useful in methods for treating CNS Lymphoma in a subject in need thereof.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for treating central nervous system lymphoma in a subject in need thereof comprising
(a) administering to the subject an effective amount of methotrexate; and
(b) administering to the subject an amount of glucarpidase that is sufficient to reduce the level of methotrexate in the subject's serum by >90%, wherein the glucarpidase is administered 10 to 48 hours following administration of methotrexate, and
wherein steps (a)-(b) are performed for 1, 2, 3, 4, 5, 6, 7, 8 or more cycles.

2. A method for reducing the toxicity associated with high dose methotrexate therapy in a subject in need thereof comprising
(a) administering to the subject an effective amount of methotrexate; and
(b) administering to the subject an amount of glucarpidase that is sufficient to reduce the level of methotrexate in the subject's serum by >90%, wherein the glucarpidase is administered 10 to 48 hours following administration of methotrexate,
wherein steps (a)-(b) are performed for 1, 2, 3, 4, 5, 6, 7, 8 or more cycles, and wherein the subject suffers from or is diagnosed with central nervous system lymphoma.

3. The method of claim 1, wherein the effective amount of methotrexate is about 3 to 10 g/m².

4. The method of claim 1, wherein the subject is suffering from or is diagnosed with diffuse large B-cell lymphoma (DLBCL) or wherein the subject is suffering from CNSL relapse.

5. The method of claim 1, wherein the central nervous system lymphoma is primary CNSL (PCNSL) or secondary CNSL (SCNSL), optionally wherein the PCNSL has developed in one or more tissues selected from the group consisting of brain parenchyma, spinal cord, meninges, cerebrospinal fluid, and eyes.

6. (canceled)

7. The method of claim 1, wherein the subject exhibits metastases in one or more tissue sites selected from the group consisting of bone marrow, testicles, paranasal sinuses, bone, retroperitoneal lymph nodes and epidural space.

8. (canceled)

9. The method of claim 1, wherein methotrexate or glucarpidase is administered subcutaneously, intravenously, intraperitoneally, intra-articularly, intra-synovially, intrasternally, intrathecally, orally, topically, transmucosally, iontophoretically, or via intracranial injection and/or wherein glucarpidase and methotrexate are administered sequentially, or separately.

10. (canceled)

11. (canceled)

12. (canceled)

13. The method of claim 1, wherein steps (a)-(b) are performed for up to 10 cycles or for 3-8 cycles.

14. (canceled)

15. The method of claim 1, wherein the amount of glucarpidase in at least one cycle is about 1800 to 2200 units or about 800 to 1200 units.

16. (canceled)

17. The method of claim 1, wherein the amount of glucarpidase in the first 2 to 4 cycles is about 1800 to 2200 units and the amount of glucarpidase after the first 2 to 4 cycles is about 800 to 1200 units; or wherein the amount of glucarpidase in the first 2 to 4 cycles is about 2000 units and the amount of glucarpidase after the first 2 to 4 cycles is about 1000 units.

18. (canceled)

19. The method of claim 1, further comprising administering to the subject an effective amount of an additional therapeutic agent that targets CNSL.

20. The method of claim 19, wherein the additional therapeutic agent that targets CNSL is selected from the group consisting of an anti-CD20 antibody, an anti-CD19 antibody, a steroid, a chemotherapeutic agent, and any combination thereof.

21. The method of claim 20, wherein the anti-CD20 antibody is rituximab, ocrelizumab, obinutuzumab, veltuzumab, ofatumumab, ibritumomab tiuxetan, ¹³¹I tositumomab, AME-133v, PRO131921, TRU-015, or GA101.

22. The method of claim 20, wherein the anti-CD19 antibody is Blinatumomab, GBR 401, Coltuximabravtansine, MOR208, MEDI-551, Denintuzumabmafodotin, Taplitumomabpaptox, XmAb 5871, MDX-1342, AFM11, or SAR3419 (huB4-DM4).

23. The method of claim 20, wherein the chemotherapeutic agent is cyclophosphamide, carmustine, etoposide, bisulfan, vincristine, procarbazine, temozolomide, cytarabine, or thiotepa.

24. The method of claim 20, wherein the anti-CD20 antibody is administered in one or more cycles with methotrexate and glucarpidase.

25. The method of claim 24, wherein each cycle occurs over (i) a three day period and (ii) comprises:
administering to the subject about 300-600 mg/m² of anti-CD20 antibody on day 1,
administering to the subject about 3-10 mg/m² methotrexate on day 2, and
administering to the subject about 800-2200 units of glucarpidase on day three.

26. The method of claim 1, wherein the subject exhibits a delay in metastatic onset and/or tumor growth after administration of MTX and glucarpidase compared to that observed in an untreated control subject diagnosed with CNSL.

27. A kit comprising MTX, glucarpidase, and instructions for treating CNSL according to the method of claim 1.

28. The kit of claim 27, further comprising an additional therapeutic agent selected from the group consisting of an anti-CD20 antibody, an anti-CD19 antibody, a steroid, a chemotherapeutic agent, and any combination thereof.


SEQUENCE LISTING (1 sequence)

SEQ ID NO: 1 - protein, 393 aa, Artificial Sequence
  Description of Artificial Sequence Synthetic polypeptide
ALAQKRDNVLFQAATDEQPAVIKTLEKLVNIETGTGDAEGIAAAGNFLEAELKNLGFTVTRSKSAGLVVGDNIVGKIKGRGGKNLLLMSHMDTVYLKGILAKAPFRVEGDKAYGPGIADDKGGNAVILHTLKLLKEYGVRDYGTITVLFNTDEEKGSFGSRDLIQEEAKLADYVLSFEPTSAGDEKLSLGTSGIAYVQVNITGKASHAGAAPELGVNALVEASDLVLRTMNIDDKAKNLRFNWTIAKAGNVSNIIPASATLNADVRYARNEDFDAAMKTLEERAQQKKLPEADVKVIVTRGRPAFNAGEGGKKLVDKAVAYYKEAGGTLGVEERTGGGTDAAYAALSGKPVIESLGLPGFGYHSDKAEYVDISAIPRRLYMAARLIMDLGAGK